(19) United States
(12) Patent Application Publication
(10) Pub. No.: US 2015/0150642 A1
(43) Pub. Date: Jun. 4, 2015

(54) Device and Method for In-Office Unsedated Tracheoesophageal Puncture (TEP)

(71) Applicants: Singapore Health Services Pte Ltd, Singapore (SG); National University of Singapore, Singapore (SG)

(72) Inventors: David Pang Cheng LAU, Singapore (SG); Chin Boon CHNG, Singapore (SG); Chee Kong CHUI, Singapore (SG)

(21) Appl. No.: 14/304,395

(22) Filed: Jun. 13, 2014

Related U.S. Application Data
(63) Continuation of application No. PCT/SG2012/000470, filed on Dec. 12, 2012.
(60) Provisional application No. 61/576,343, filed on Dec. 15, 2011.

(51) Int. Cl. A61B 19/00; A61B 17/34

(52) U.S. Cl. CPC A61B 19/46; A61B 17/3468; A61B 17/3415; A61B 17/3476; A61B 17/3494; A61B 19/54; A61B 2017/3409; A61B 2019/461; A61B 2019/464; A61B 2019/5437

(57) ABSTRACT

Various embodiments of the present disclosure are directed to apparatuses, devices, and procedures for unsedated in-office tracheoesophageal puncture (TEP) using transnasal esophagoscopy (TNE) and a measurement cannula, referred to as a TEP measurement and insertion device (MAID or TEP MAID). In some embodiments, a MAID is configured for use with a mini-tracheostomy kit; in other embodiments, a MAID is configured to be carried and automatically or semi-automatically deployed, for instance, by a handheld mechatronic device or an automated device such as a medical robot that carries an automated tracheoesophageal puncture and voice prosthesis insertion end effector. In accordance with an aspect of the present disclosure, a MAID has a body that carries or includes indicators, indicia, or markings that facilitate or enable substantially direct and/or immediate estimation, identification, or measurement of a tissue extent or thickness corresponding to or across a tracheoesophageal fistula (e.g., a tracheoesophageal dividing wall thickness), and selection and insertion of a voice prosthesis, such as a non-indwelling voice prosthesis from Blom-Singer. In certain embodiments, a MAID includes at least an outer measurement cannula and an inner adaptor cannula configured to fit over or matingly engage with a dilator, such as a short dilator provided in Portex's mini-tracheostomy kit.

### CROSS-REFERENCE TO RELATED APPLICATIONS

This application is a continuation of International Application Number PCT/SG2012/000470, with an international filing date of 12 Dec. 2012, which was published in English under PCT Article 21(2) as International Publication Number WO 2013/089644, and which is incorporated by reference herein in its entirety.

### TECHNICAL FIELD

The present disclosure relates generally to devices and methods for unsedated in-office tracheoesophageal puncture (TEP) using transnasal esophagoscopy (TNE). More particularly, particular aspects of the present disclosure are directed to a TEP measurement and insertion device (MAID, or TEP MAID) having a body that carries or includes identifiers or markings, which can facilitate or enable the immediate estimation, identification, or measurement of a tissue extent or thickness corresponding to or across a tracheoesophageal fistula (e.g., a tracheoesophageal dividing wall thickness), and/or selection and insertion of a voice prosthesis (e.g., a non-indwelling Blom-Singer voice prosthesis). In accordance with an embodiment of the disclosure, the MAID includes at least an outer measurement cannula and an inner adaptor cannula that fits over a dilator, such as a short dilator provided by Portex's mini-tracheostomy kit. Additional aspects of the present disclosure are directed to an automated or semi-automated device (e.g., an electromechanical or pneumatic handheld device or robotic manipulator) configured for unsedated in-office TEP and/or voice prosthesis insertion procedures, which can carry a MAID and which can further carry and controllably deploy a transesophageal puncture element, member, device, or tool and/or a voice prosthesis insertion element, member, device, or tool.

### BACKGROUND

Loss of voice following total laryngectomy (TL) can result in disabling psychosocial and economic consequences for the patient. Fortunately a number of options are available to restore voice, including the mechanical larynx, esophageal speech, and tracheoesophageal puncture (TEP). Of these, TEP speech provides the best fluency rates and is the gold standard for voice rehabilitation.

TEP can be performed as a primary procedure at the time of TL, or later as a secondary procedure. Most studies show somewhat better voice results following primary procedure with 65-96% of patients continuing to use the prosthesis for a year or more, compared to 69-83% following secondary TEP. This may be partly because secondary TEP is often performed in difficult circumstances, for example if primary surgery is extensive and requires free flap or gastric pull-up reconstruction, after heavy neck irradiation, or when alternative voicing techniques including primary TEP have failed. Some surgeons routinely perform secondary TEP to optimize placement of the puncture and prevent potential complications such as cervical cellulitis, mediastinitis and salivary leakage, which may adversely affect healing of the TL site.

Classically, secondary TEP is performed under general anesthesia with rigid esophagoscopy, but there is an increased trend towards unsedated in-office TEP. In-office TEP avoids risks of general anesthesia and those associated with rigid esophagoscopy such as esophageal perforation, and oral injury. It enables cannulation of the esophagus in patients with limited neck extension or stenosis of the neopharynx, allows rapid recovery and decreases the need for patient monitoring. Performing the procedure in an outpatient, office-based setting can also reduce cost.

Several techniques for unsedated in-office secondary TEP have been described. Desyatnikova et al carried out blind puncture using a 16-gauge needle. An esophageal dilator swallowed by the patient provided tactile feedback to confirm needle entry into the esophagus, and also protected the posterior esophageal wall from trauma by the needle. With the needle in place a guide-wire was passed through it after which the esophageal dilator was withdrawn. A separate dilator passed over the guide-wire was followed by the prosthesis. Direct laryngoscopy provided some degree of visualization.

Erenstein and Schouwenburg also performed blind puncture but used an endotracheal tube through the mouth to dilate the esophagus. A flexible nasopharyngoscope passed into the endotracheal tube trans-illuminated the posterior tracheal wall to indicate its position. With the cuff inflated to hold the endotracheal tube in position, a trocar was passed through the posterior tracheal wall into the esophagus and the endotracheal tube. A guide-wire passed through the trocar was brought out of the mouth through the endotracheal tube. A dilator placed on the tracheal end of the guide-wire was used to enlarge the fistula by withdrawing the guide-wire through the mouth. The prosthesis was then inserted through the dilated tract.

U.S. Pat. No. 5,078,743 describes a safe and easy method of inserting voice prosthesis in a patient presenting with minimal complications. The said voice prosthesis preferably used is Blom-Singer device. The said implantation procedure employs steps like insertion of graduated catheter and introduction of cannula along a guide wire is performed. A Seldinger needle is used to puncture the tracheoesophageal tract for the introduction of outer cannula. Further dilation of the esophageal tract, removal of measuring cannulae is included.

Transnasal esophagoscopy (TNE) guided TEP was first described in 2001 by Belafsky et al. In their case report, TNE allowed the replacement of a poorly placed TEP under direct vision. Subsequently, Bach et al 8 described their technique in 2003. In their method, TNE was used to visualize the puncture site under local anesthesia. The puncture position was marked with a 22-gauge needle, and a stab incision was made along the same tract and widened with a hemostat. Following this, a TEP prosthesis was placed. Two additional series on TNE-guided TEP were identified in the literature.

In 2007, Doctor reported 11 patients undergoing TNE-guided TEP following TL. The success rate for secondary TEP placement was 91%. One patient was complicated by bleeding from the puncture site which was arrested with silver nitrate cautery. Doctor described using a straight needle to guide placement of the puncture site and performed the puncture with a size 11 blade. In 2009, LeBert studied 39 patients undergoing TNE-guided TEP. The overall success rate was 97% and included patients who had undergone TL (64% of cases), TL with partial pharyngectomy (21% of cases), and microvascular flap reconstruction (36% of cases). Radiotherapy or cricopharyngeal myotomy did not significantly impact the success of TEP placement, complications associated with TEP placement, TEP prosthesis usage or speech intelligibility. Le Bert described making the puncture directly with a size 11 blade after visualizing indentation of the posterior tracheal wall by ballottement.

The main advantage of TNE-guided TEP over earlier techniques is the ability to visualize the esophageal lumen. An additional benefit is that the discomfort of swallowing a dilator or endotracheal tube is avoided. Visualizing the esophagus during TEP has the following advantages: 1) False tract formation can be prevented. 2) Viewing the needle tip within the esophagus helps minimize trauma to the posterior esophageal wall. Care must still be taken during initial needle insertion as the anterior esophageal wall can tent up against the posterior wall despite air insufflation of the esophagus. However, trauma from the initial insertion is usually insignificant, and once the needle tip is seen intra-luminally, its position can be controlled. 3) The puncture can be directed to a more open part of the esophageal lumen. Avoiding constricted areas may improve airflow during subsequent voicing which may have implications on improving voice outcome. To achieve optimal needle placement, the puncture may need to be directed sideways as esophagus and trachea are sometimes off-centre in relation to the sagittal plane. 4) Finally, anatomical distortion resulting from reconstruction may be easier to negotiate using flexible rather than rigid esophagoscopy.

In 2010, our group described the use of the mini-tracheostomy kit to perform TNE-guided TEP. This provided additional benefits: Dilating rather than incising tissue minimizes bleeding and trauma to surrounding tissue. The Seldinger technique which dilates tissue over a guide-wire practically eliminates the risk of creating a false passage, which can still occur when inserting a tube or prosthesis through an incision without guide-wire. The downward-angled Tuohy needle helps ensure the guide-wire is directed downwards into the esophagus, and the dilators in the mini-tracheostomy kit are curved and softened which minimizes soft tissue trauma. These features allow the procedure to be completed safely and rapidly within several minutes. Finally, nearly all instruments within the kit are utilized, making it a well contained unit and minimizing wastage.

One shortcoming common to the in-office techniques is that voice prosthesis is usually not inserted at the time of fistula tract creation. Firstly, because it is difficult to measure the exact length of the prosthesis required at the time of tract creation, and secondly because the tract requires time to heal, particularly if the tract has been created traumatically or by incising tissue. As a result, patients often require a week or two of stenting with a nasogastric tube or catheter, which is uncomfortable and delays the voicing process. There is a need for (1) relatively a-traumatic means to create a fistula, and (2) ability to insert the voice prosthesis accurately and immediately at the time of initial puncture.

Current methods for TEP either require general anesthesia or if performed unsedated are uncomfortable procedures and risk injury to the esophagus. There is a need for a simpler, safer method to perform unsedated TEP. A device for rapid TEP in unsedated patients with minimal risk of complications such as posterior esophageal wall injury would be a significant advance in voice restoration following total laryngectomy.

### SUMMARY

Various embodiments of the present disclosure are directed to apparatuses, devices, and procedures for unsedated in-office tracheoesophageal puncture (TEP) using transnasal esophagoscopy (TNE) and a measurement cannula, which is referred to herein as a TEP measurement and insertion device (MAID or TEP MAID). In some embodiments, a MAID is configured for use with a mini-tracheostomy kit; in other embodiments, a MAID is configured to be carried and automatically or semi-automatically deployed, for instance, by a handheld mechatronic device or an automated device such as a medical robot that carries an automated tracheoesophageal puncture and voice prosthesis insertion end effector.

In accordance with an aspect of the present disclosure, a MAID has a body that carries or includes indicators, indicia, or markings that facilitate or enable substantially direct and/or immediate estimation, identification, or measurement of a tissue extent or thickness corresponding to or across a tracheoesophageal fistula (e.g., a tracheoesophageal dividing wall thickness), and selection and insertion of a voice prosthesis, such as a non-indwelling voice prosthesis from Blom-Singer.

In certain embodiments, a MAID includes at least an outer measurement cannula and an inner adaptor cannula configured to fit over or matingly engage with a dilator, such as a short dilator provided in Portex's mini-tracheostomy kit. In accordance with related aspects of the present disclosure, a procedure for TEP uses TNE to visualize the esophageal lumen, and utilizes portions of a Seldinger Portex Minitracheotomy Kit to create a fistula. In an embodiment, the procedure includes the following: (a) a 16-gauge needle is inserted through the posterior wall of the trachea into the esophagus and a Seldinger guide-wire is passed through it; (b) serial curved dilators passed over the guide-wire allow creation of a tract; (c) a 4-mm internal diameter mini-tracheostomy cannula passed over a dilator is positioned between trachea and esophagus; (d) a nasogastric tube is then passed through the mini-tracheostomy tube into the esophagus and left in place for a week while the fistula tract matures; (e) a measurement cannula is left behind and an inner adaptor cannula is removed. Detectable, visible, or external markings carried by the measurement cannula are visualized within the esophageal lumen (e.g., using TNE), thereby allowing the appropriate prosthesis length to be determined; and (f) a selected prosthesis is then inserted directly through the cannula.

In accordance with another aspect of the present disclosure, the procedure is carried out under transnasal esophagoscopy (TNE) control and without general anesthesia or sedation. TNE dilates the esophageal lumen by air insufflations and allows clear visualization. This reduces the risk of false tract formation and posterior esophageal wall injury, and enables the puncture to be well aligned (the trachea and esophagus may not be directly in line). Additionally, it facilitates measurement of the tracheoesophageal dividing wall thickness by visualizing the intra-luminal part of the MAID. Visualization also ensures that the intra-luminal flange of the voice prosthesis is correctly positioned during and after placement.

In accordance with a further aspect of the present disclosure, the puncturing of the tissue and/or the insertion of an appropriate voice prosthesis can be performed using an automated or semi-automated device, such as a handheld electromechanical (e.g., mechatronic) device or a fully automated robotic manipulator that is configured for carrying or deploying a MAID, and which is further configured for carrying and controllably deploying at least one of (a) a transesophageal puncture element, member, device, or tool; and (b) a voice prosthesis insertion element, member, device, or tool.

### BRIEF DESCRIPTION OF THE DRAWINGS

FIG. 1 is an exploded view of a tracheoesophageal puncture (TEP) measurement and insertion device (MAID) in accordance with an embodiment of the present disclosure.

FIG. 2 is an illustration of a MAID having an adaptor cannula mounted on a short dilator in accordance with an embodiment of the present disclosure.

FIG. 3A-3G is a sequence of images depicting a THE visualized MAID-based TEP procedure in accordance with an embodiment of the present disclosure.

FIG. 4A is an exploded view of a handheld mechatronic TEP tool configured for carrying a MAID and a puncture tool in accordance with an embodiment of the present disclosure.

FIG. 4B is a schematic illustration of an insertion tool configured for inserting a voice prosthesis through a MAID and deploying the voice prosthesis in a patient in accordance with an embodiment of the present disclosure.

FIG. 5A is a schematic illustration of a handheld mechatronic tracheoesophageal puncture and voice prosthesis insertion tool in accordance with an embodiment of the present disclosure.

FIG. 5B is a schematic perspective illustration of internal portions of a main body of a handheld mechatronic tracheoesophageal puncture and voice prosthesis insertion tool in accordance with an embodiment of the present disclosure.

FIG. 5C is a side view corresponding to portions of a measurement cannula and an associated main body of a handheld mechatronic tracheoesophageal puncture and voice prosthesis insertion tool following a puncture stroke and after puncture tool removal in accordance with an embodiment of the present disclosure.

FIG. 5D is a side view corresponding to portions of a main body of a handheld mechatronic tracheoesophageal puncture and voice prosthesis insertion tool following an insertion stroke, just after voice prosthesis insertion in accordance with an embodiment of the present disclosure.

FIG. 5E is a schematic representation of a graph of forces on an actuator and sensed by a force sensor during a TEP procedure.

FIG. 6A is an exploded view of a macro-micro positioning system including an automated TEP end effector configured for carrying a MAID and a puncture tool in accordance with an embodiment of the present disclosure.

FIG. 6B is a perspective view of an embodiment of the TEP end effector of FIG. 6A

FIG. 6C is a schematic illustration of a prototype automated macro-micro positioning system carrying an automated tracheoesophageal puncture and voice prosthesis insertion end effector, and a display screen capture of a computer simulation corresponding thereto in accordance with an embodiment of the present disclosure.

### DETAILED DESCRIPTION

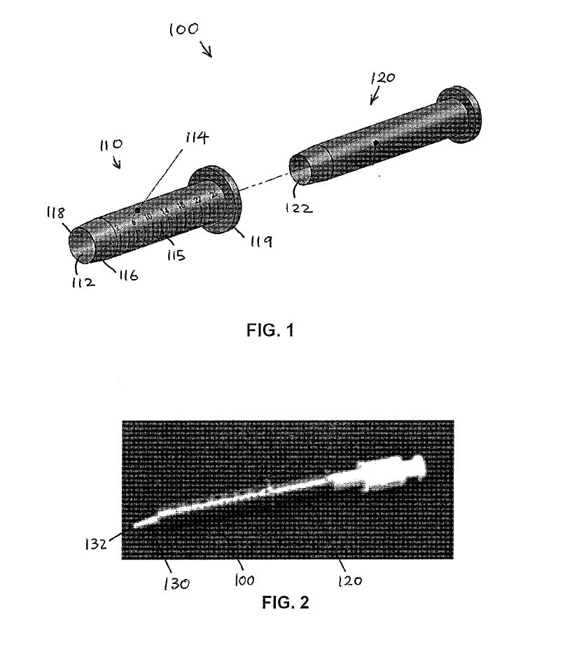

FIG. 1 is an exploded view of a MAID 100 or TEP MAID in accordance an embodiment of the present disclosure; and FIG. 2 is an illustration of such a MAID 100 with an adaptor cannula 120 mounted on a short dilator 130 according to an embodiment of the present disclosure. In an embodiment, a MAID includes at least an outer measurement cannula 110 and an inner adaptor cannula 120 configured to fit over or engage (e.g., matingly engage) with portions of a dilator 130 such as a short dilator provided by Portex's mini-tracheostomy kit.

Particular aspects of the MAID that facilitate enable essentially or substantially direct or immediate prosthesis insertion at the time of fistula tract creation are described in the following.

The outer measurement cannula 110 of the MAID 100 is configured with a cylindrical or tubular shape with an inner lumen 112 defining an inner diameter sized to enable the outer measurement cannula 110 to slide on or off a dilator 130 and/or an adaptor cannula 120. The inner diameter is also sized to allow a voice prosthesis 140 to pass through easily. Thus in one embodiment, to permit easy passage of a standard-sized 16Fr voice prosthesis having a diameter of 5.3 millimeters (mm) such as a Blom-Singer® voice prosthesis (available from InHealth Technologies), the outer measurement cannula 110 is configured with an inner diameter of 6.5-6.6 mm. The surface of the inner lumen 112 is smooth, so as to facilitate easy sliding on or off of the dilator 130, the adaptor cannula 120 and the voice prosthesis 140. The outer diameter of the outer measurement cannula 110 in one embodiment is 7.5 mm wide. This thickness of the outer measurement cannula 110, together with the use of medical grade titanium, stainless steel or similarly robust plastic, is found sufficient to ensure that the outer measurement cannula 110 does not deform during the insertion procedure.

In representative implementations in accordance with particular embodiments of the present disclosure, a MAID 100 can have an inner diameter of approximately 6.5 mm to accommodate a voice prosthesis having a 16Fr flange size, or an inner diameter of approximately 7.75 mm to accommodate a voice prosthesis having a 20Fr flange size. In alternative embodiments, a MAID can exhibit another inner diameter. In general, a MAID outer diameter should be as close to its inner diameter as possible, as long as the MAID can maintain or retain its shape within a fistula. In a representative implementation in which the MAID includes or is made of stainless steel, a MAID thickness is approximately 0.5 mm. If a weaker material is used, MAID wall thickness could be increased accordingly.

The outer surface 114 of the outer measurement cannula is smooth with a 5° bevel 116 or chamfer extending for about 5 mm from a leading edge or distal end 118 of the outer measurement cannula. This advantageously facilitates reduction of friction and tissue trauma during the insertion procedure. The outer measurement cannula 110 is configured to be long enough so that, in use, it can bridge the tracheoesophageal dividing wall between the esophagus and the trachea. It is however not desirable to make the outer measurement cannula 110 so long that it becomes difficult to pass the voice prosthesis 114 through the inner lumen 112 of the outer measurement cannula 110. For example, in one embodiment, the outer measurement cannula 110 is 40.0 mm long, and in an alternative embodiment the outer measurement cannula 110 in assembly with the adaptor cannula 120 is 33.3 mm long. Alternatively, the MAID 100 is longer than 40.0 mm. The length of the MAID 100 should be sufficient for the MAID 100 to bridge the trachea-esophageal dividing wall, and sufficient for (a) visualization of MAID indicia or markings 115 within the esophageal lumen by way of TNE; and (b) visualization of MAID indicia or markings 115 external to the patient.

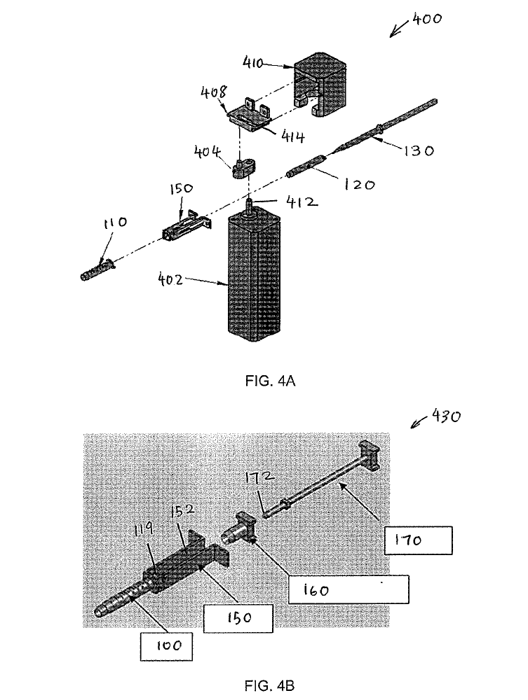

The MAID 100 can be further provided with a handle or holder 150 at its proximal end 119 as shown in FIG. 4B. This can be a light-weight extension coupled to the proximal end 119 of the outer measurement cannula 110, that is, the handle 150 can be located at the end of the outer measurement cannula furthest away from the beveled end 118 of the outer measurement cannula 110. The handle 150 facilitates easier handling and manipulation of the outer measurement cannula 110, for example, during removal of the dilator 130 and insertion of the voice prosthesis 140.

The outer measurement cannula 110 is provided on its outer surface 114 with indicators, markings, indicia, engravings or visible, perceptible or detectable features (collectively referred to as “markings” 115 for convenience) distributed along the length of the outer measurement cannula 110 at regular intervals or spacings. Neighboring or adjacent markings can be spaced, for example, 2 mm apart from one another. The markings 115 provide an accurate and easy means for gauging or measuring the width of the tracheoesophageal dividing wall. Measurement can be performed by visualizing the external and intra-luminal markings and subtracting or taking the difference therebetween. The outer measurement cannula 110 can made suitable for sterilization for multiple usage or disposable, depending on the material of construction. Therefore, in the case of the former, the markings 115 are preferably engraved so that they cannot be erased, smudged, or otherwise worn off in the course of multiple usage and repeated sterilization.

According to another aspect of the present disclosure, the MAID 100 includes an adaptor cannula 120 configured for slidable and releasable engagement with the outer measurement cannula 110. The adaptor cannula 120 is a cylindrical or tubular sleeve with an outer diameter sized for slidable engagement with the inner lumen 112 of the outer measurement cannula 110. At the same time, a lumen 122 defining the inner diameter of the adaptor cannula is sized to permit smooth passage of the dilator 130. The provision of the adaptor cannula 120 advantageously enables an outer measurement cannula 110 that is sized to accommodate a standard size voice prosthesis to be used with the widest dilator in Portex's mini-tracheostomy kit. The adaptor cannula 120 is configured to fit over or matingly engage with the dilator 130, and within the inner lumen 112 of the outer measurement cannula 110. The adaptor cannula 120 thus facilitates a smooth transition between the dilator 130 and outer measurement cannula 110, and enables the overlying outer measurement cannula 110 to slide off more easily than if the outer measurement cannula 110 is directly over the dilator 130. A less desirable alternative to the provision of the adaptor cannula 120 is the use of a dilator that is wider than the widest dilator available in Portex's mini-tracheostomy kit, as a wider dilator will create more tissue trauma or create a fistula too large for a good fit with the selected voice prosthesis.

A specially designed plunger 170 is provided for use with the MAID 100 to introduce the voice prosthesis 140 through the outer measurement cannula 110. The plunger 170 can be essentially identical or analogous to a plunger described below with reference to FIG. 4B. The plunger 170 includes a blunt end 172 to avoid damaging the voice prosthesis. It is designed to allow controlled introduction of the voice prosthesis by a push-pull action. Once the intra-luminal flange 142 of the prosthesis unfolds in the esophageal lumen and is brought back flush with the esophageal mucosa, the outer measurement cannula 110 is withdrawn over the prosthesis 140, which is kept relatively immobile by pressure from the plunger 170. This ensures that the flanges open at either end of the tracheoesophageal fistula and not within it.

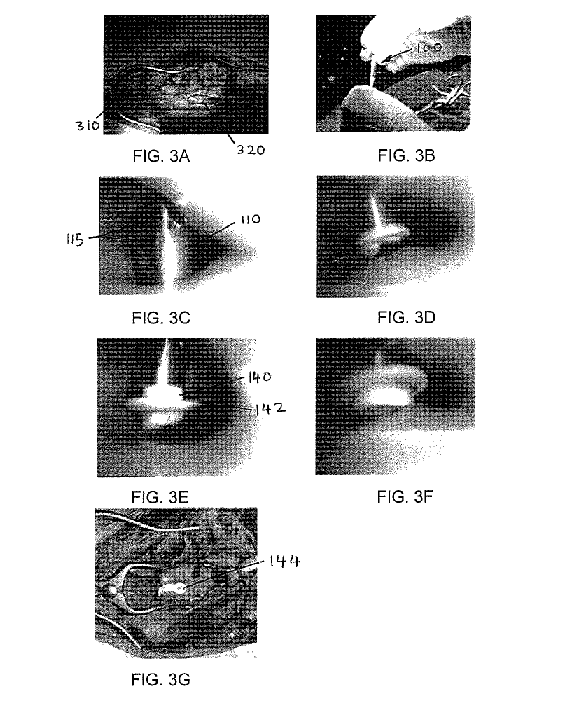

In a patient who has undergone laryngectomy, the device and method according to one embodiment of the present disclosure provides an improved way of placing a voice prosthesis 140 in a surgically created fistula between the posterior tracheal wall and the anterior esophageal wall, at the time of first creating the initial puncture for forming the fistula with minimal tissue trauma. A THE visualized MAID-based TEP procedure in accordance with an embodiment of the present disclosure is described with reference to FIGS. 3A-3G, a series of images taken during an animal experiment. In an embodiment, as shown in FIG. 3A, the procedure involves insertion of a Seldinger guide-wire 310 through a stoma, through the posterior tracheal wall 320, up the esophagus, and out through the nasal passage (not shown).

In FIG. 3B, a MAID 100, assembled with a dilator 130, is passed over the guide-wire 310. The distal end 132 of the dilator or puncture tool 130 which extends from the distal end 118 of the outer measurement cannula 110 is tapered and thus shaped to dilate the tracheoesophageal puncture through which the guide-wire 310 runs, thereby creating a fistula of a desired size. The use of the adaptor cannula 120 between the outer measurement cannula 110 and the dilator 130 enables the use of a smaller or narrower dilator 130 than is possible with conventional means, and thereby reducing unnecessary tissue trauma for the patient.

In practically the same motion as the formation of the fistula by the dilator 130, the outer measurement cannula 110 passes through the fistula and into the esophageal lumen unobtrusively, owing to the bevel 116 provided at the distal end 118 of the outer measurement cannula 110, and to the smooth transition from the dilator 130 to the outer surface 114 of the outer measurement cannula 110. The dilator 130, as well as the adaptor cannula 120 if used, can be removed from the outer measurement cannula 110 by withdrawing it from the proximal end 119 of the outer measurement cannula 110.

At this stage, as shown in FIG. 3C, a first set of markings 115 or some of the markings on the outer measurement cannula 110 can be visually detectable by a visual inspection device, such as a transnasal esophagoscopy (TNE) device or a camera, disposed in the esophageal lumen. Other means of identifying, detecting, or capturing intra-luminal identifiers, markings, or indicia carried by the outer measurement cannula can also be used. At the same time, a second set of markings 115 or some other of the markings on the part of the outer measurement cannula 110 on the trachea side of the fistula (and therefore external to the patient's body) can be visible or visually detectable. The length of the fistula can therefore be determined by taking the difference between the two sets of markings 115.

Having determined the length of the fistula, a voice prosthesis 140 of an appropriate or suitable dimension, size, or length is selected. One exemplary voice prosthesis includes a one-way valve in a channel having an internal or intra-luminal flange 142 and an external or extra-luminal flange 144. Voice prostheses may come in various standard sizes, and one may be selected so that the internal flange 142 and the external flange 144 will grip the tracheoesophageal wall around the fistula, without the voice prosthesis 140 getting dislodged or causing excessive discomfort to the patient. The selected voice prosthesis can be loaded or fitted in the now empty inner lumen 112 of the outer measurement cannula 110 with the flanges folded therein and the inner flange 142 nearer the distal end 118 of the outer measurement cannula 110. Loading of the voice prosthesis can be facilitated by a prosthesis loader 160, an example of which is shown in FIG. 4B. The outer measurement cannula 110 which has remained disposed through the fistula provides a channel by means of its inner lumen 112 for the delivery of the voice prosthesis.

A plunger 170, such as one illustrated in FIG. 4B, can then be used to push the voice prosthesis 140 along the inner lumen 112 of the outer measurement cannula 110, until the inner flange 142 of the voice prosthesis emerges from the distal end 118 of the outer measurement cannula into the esophageal lumen, as shown in FIGS. 3D-3E. By keeping the plunger 170 stationary relative to the outer measurement cannula 110 while withdrawing the outer measurement cannula 110 from the esophageal lumen, the inner flange 142 of the voice prosthesis can be brought to abut the anterior esophageal wall, as shown in FIG. 3F. When the inner flange 142 of the voice prosthesis is substantially flush with the anterior esophageal wall, the plunger 170 is used to apply pressure on the voice prosthesis 140 so that the voice prosthesis 140 remains in place in the fistula while the outer measurement cannula 110 slides out of the fistula and is thereby removed. As can be appreciated, the outer measurement cannula 110 is inserted into the patient's body when the initial puncture of the TEP is made, and the outer measurement cannula 110 is removed only after the voice prosthesis 140 has been placed in its final position with its inner flange 142 flush with the anterior esophageal wall. The handle 150 of the outer measurement cannula and the plunger 170 cooperate to facilitate this process. FIG. 3G shows the voice prosthesis in place with the external flange resting on the posterior tracheal wall when the outer measurement cannula 110 and the plunger 170 are removed. Embodiments of the method and device of the present disclosure thus obviate the need for repeated inserted and removal of a device through the fistula from the time of initial puncture to the completion of the placement of a voice prosthesis, and accordingly significantly decreases the discomfort suffered by the patient. The voice prosthesis 140 can thus be placed in the fistula at the same time as the initial puncture or dilation for forming the fistula, and the patient does not have to suffer delay in the voicing process.

In accordance with an embodiment of the present disclosure, an automated or semi-automated TEP tool 400 can be configured for carrying a MAID 100 and a dilator or puncture tool 130, and can be further configured for creating a TEP and trans-luminal deployment of the MAID 100 by way of rapid constrained puncture tool displacement.

FIG. 4A is an exploded view of a handheld TEP tool 400 configured for carrying a MAID 100 and a puncture tool 130 in accordance with an embodiment of the present disclosure. In an embodiment, the TEP tool 400 includes: a motor 402 that can also serve as a handle; a motor coupling 404; a MAID holder 150; a measurement cannula 110; an adaptor cannula 120; a puncture tool 130; a slide 408; a cover 410. An output shaft 412 of the motor can be operably coupled to the slide 408 via the motor coupling 404 such that the torque generated by the motor 402 is converted to a linear force for driving the slide 408. This can be implemented by coupling the motor coupling 404 to a slot 414 provided in the slide 408. In this fashion the motor can be configured to drive the puncture tool 130 carried by the MAID 100 at a desired speed to create the TEP. Once a tract or fistula is created, the puncture tool 130 can be removed, and an appropriate voice prosthesis 140 can be inserted through the MAID 100.

FIG. 4B is a schematic illustration of an insertion tool 430 that can be carried by a handheld TEP for inserting a voice prosthesis 140 through a MAID 100 and deploying the voice prosthesis 140 in a patient or subject in accordance with an embodiment of the present disclosure. In an embodiment, the insertion tool 430 includes an elongate plunger 170; a prosthesis loader 160 configured to carry a voice prosthesis 140; a handle 150; and a MAID 100. In response to a displacement force applied along the length of the plunger 170, the plunger 170 can push the prosthesis loader 160 in which the voice prosthesis 140 resides along a channel 152 in the handle, and can further push the voice prosthesis itself through an inner opening (not shown) at the proximal end 119 of the MAID and into the patient. A forward stroke distance of the plunger 170 can be essentially or approximately equal to or correlated with a forward stroke distance of the puncture tool 130, such that the voice prosthesis 140 is inserted within and appropriately deployed across the tracheoesophageal fistula.

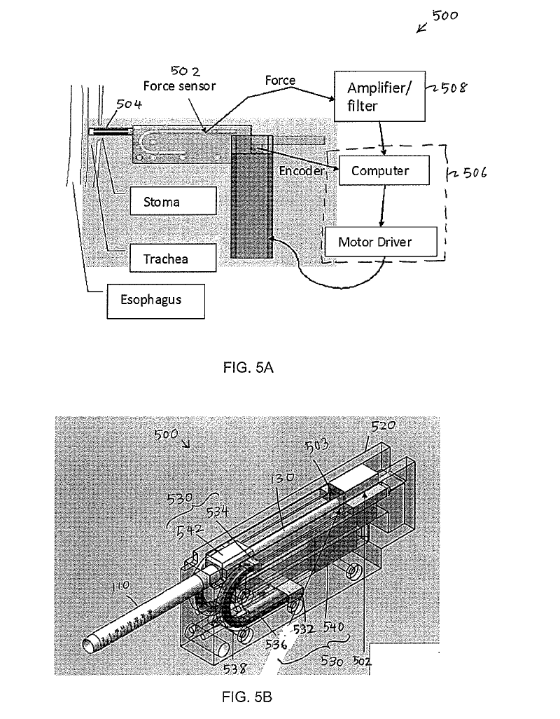

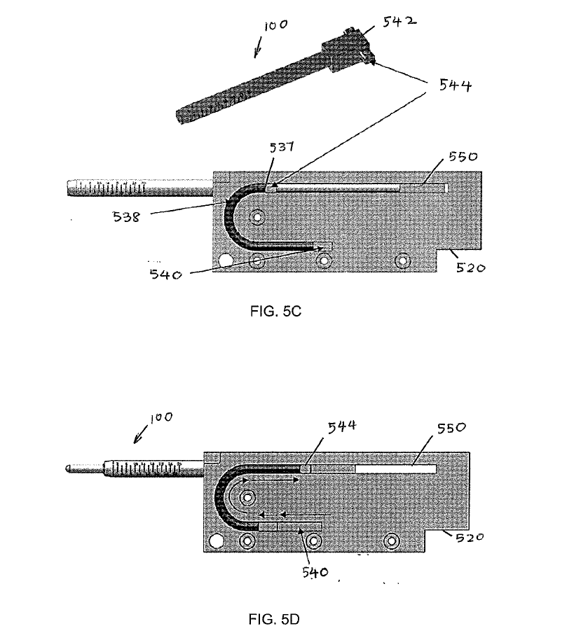

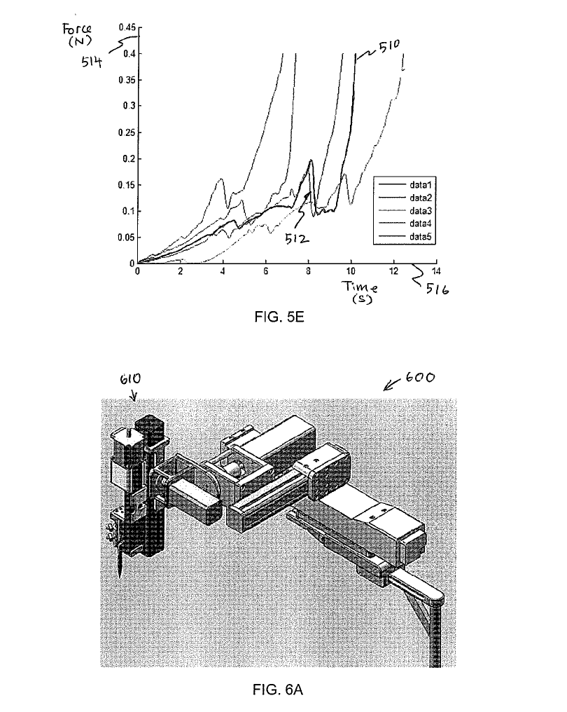

FIG. 5A is a schematic illustration of a handheld mechatronic tracheoesophageal puncture and voice prosthesis insertion tool 500 in accordance with an embodiment of the present disclosure. The handheld mechatronic device 500 includes a single axis mechanism actuator that can be coupled with interchangeable tools or devices for performing tracheoesophageal puncture and voice prosthesis insertion, while allowing the voice prosthesis to be installed at the same time as the creation of the tracheoesophageal fistula, that is, without requiring an intervening week or two for the fistula to heal between tracheoesophageal puncture and voice prosthesis insertion. The handheld mechatronic tool or device 500 of FIG. 5A can be provided with a force sensor 502 configured to sense reactive forces on the actuator 504, and circuitry to use this feedback for further controlling the actuator through a computer-controllable motor controller 506 operably coupled to the actuator.

According to one embodiment of the handheld mechatronic device 500, force data from the force sensor 502 is collected. By passing the force data or force feedback signal through a suitable amplifier and filter circuit 508, it is possible to identify the point when a differentiation of the force data rises before a predetermined threshold 512, as schematically illustrated in a plot 510 of force data 514 against time 516 in FIG. 5E. This point is taken to be indicative of a puncture event, that is, when a puncture tool carried on the actuator first makes a puncture or breaks through the posterior tracheal wall but before it reaches the anterior esophageal wall. Advantageously, the handheld mechatronic device 500 can be configured with force sensing and control, so that upon sensing the puncture event, the puncture tool 130 that is carried on the actuator is automatically halted and refracted. The puncture tool 130 can then be extended at a slower speed until the puncture tool 130 breaks through the anterior esophageal wall. Overextension of the puncture tool 130 is thereby avoided, and trauma to the posterior esophageal wall is thus minimized. Previously, although it is known that tissue deformation can be reduced by increasing the velocity of the puncture tool 130, making the puncture at high speed was avoided for fear of causing trauma to the posterior esophageal wall as the esophageal lumen is typically very narrow in the range of 10 to 20 mm in diameter. With the force sensing and control capabilities as proposed in the present disclosure, a cleaner fistula can be formed by driving the puncture tool 130 at a desirably higher speed without increasing the risk of causing trauma to the posterior esophageal wall.

Taking into consideration that the esophageal lumen is typically between approximately 10-20 mm in diameter, in order to create a fistula within a tracheoesophageal wall in a manner that avoids having the puncture tool travel significantly beyond the tracheoesophageal wall and thus avoids undesirable tissue damage, the puncture tool should be driven at a high speed (e.g., between approximately 2 mm/s and 15 mm/s, for instance, about 7.5-12.5 or 10 mm/s) and force. The temporal window within which puncture tool travel must be stopped can be less or significantly less than one second. A sensor-based feedback system, such as the force sensing system described above, or an optical sensor-based feedback system (e.g., configured to detect a change in a reflected illumination condition within the esophageal lumen as a result of puncture tool entry into the lumen, using a puncture tool having at least one reflective or highly reflective portion and detection of reflected illumination by way of TNE), or another type of sensor-based feedback system configured to terminate the displacement of the puncture tool must successfully stop puncture tool displacement within an appropriate temporal window to avoid undesirable tissue damage.

In one embodiment, puncture detection can be carried out by differentiating the force data obtained by the force sensor 502. When the difference rises above a preset threshold 512, it is identified as a puncture event and a retraction or braking mechanism activates to prevent overextension of the puncture tool. In on embodiment, a braking and retraction mechanism is directly actuated with the use of a separate motor stage to retract the puncture tip 132 through the measurement cannula 110 upon triggering.

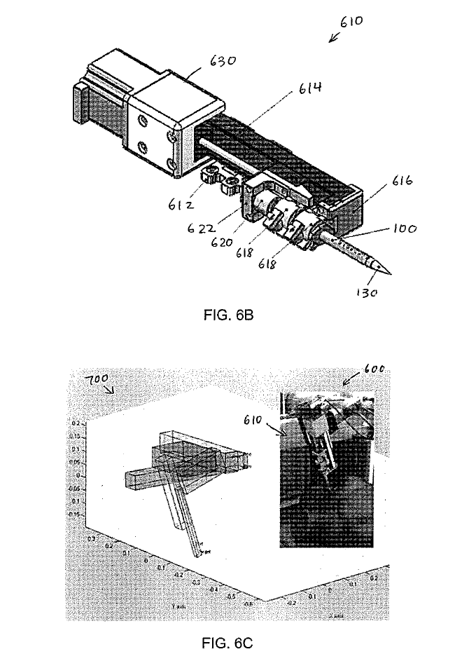

As only a single direction of force sensing is required, a pressure sensor or strain gauge can be embedded on the surface of the mounting that is directly in contact with the puncture tool 130 from the aft 503. Only the puncture tool 130 and the measurement cannula 110 would have to be removed for sterilization and can be easily removed by undoing a clamp 618, such as one shown in FIG. 6B.

Fail safe features can also be built into the system by physically limiting the maximum insertion depth of the puncture tool and preventing gross over-insertion or using software based methods of stopping the insertion upon detection of excessive force on the puncture tool tip 132.

FIG. 5B is a schematic perspective illustration of internal portions of a main body 520 of a handheld mechatronic tracheoesophageal puncture and voice prosthesis insertion tool 500 in accordance with an embodiment of the present disclosure, which indicates aspects of a retraction mechanism 530 that facilitates simultaneous insertion tool displacement for voice prosthesis insertion and deployment, and retraction or withdrawal of a MAID 100 from the tracheoesophageal fistula. A retraction mechanism 530 includes a first or lower terminal portion 532; a second or upper terminal portion 534; and a set of flexible tubing/wire/linkage elements 536 that extend between the first terminal portion 532 and the second terminal portion 534. The linkage element(s) 536 of the refraction mechanism can be disposed along or within a U-shaped channel or guide 538. During forward travel of an insertion tool such as a MAID 100 configured to carry and displace a device such as a voice prosthesis 140 for insertion into a patient, the insertion tool will engage with a displaceable force transfer member 540. Alternatively, the insertion tool can include the force transfer member 540. As the forward stroke pushes the insertion tool forward to insert and deploy the voice prosthesis in the patient, the force transfer member 540 pushes against the first terminal portion 532 of the retraction mechanism, forcing the linkage element(s) 536 to travel along the U-shaped channel 538. This force is transferred along the flexible tubing 536 within the U-shaped channel to the retraction mechanism's second terminal portion 534 and a measurement cannula interface or receiver 542 from which the measurement cannula 110 extends forward beyond or external to the main body 520 of the handheld mechatronic tool 500. Such force transfer displaces the measurement cannula interface 542 and the measurement cannula 110 in the opposite direction of insertion tool travel during the forward stroke. Thus, the insertion tool can travel forward through and beyond the body of the measurement cannula, concurrent with measurement cannula travel in a direction opposite to the direction of insertion tool travel. A voice prosthesis can therefore be inserted and deployed in the patient simultaneous with measurement cannula withdrawal from the patient.

FIG. 5C is a side view corresponding to portions of a measurement cannula 110 and an associated main body 520 of a handheld mechatronic tracheoesophageal puncture and voice prosthesis insertion tool 500 following a puncture stroke and after puncture tool removal in accordance with an embodiment of the present disclosure. As indicated in FIG. 5C, also referring to FIG. 5B in some embodiments, the second terminal portion 534 of the retraction mechanism 530 includes a set of guide rails 544 that couple to or form a portion of the measurement cannula interface 542. The guide rail(s) 544 can be configured for displacement within a set of channels 550 disposed along the main body's length. For instance, the guide rail(s) 544 can be configured to slide within a side upper slot 550 of the main body 520. During puncture, the measurement cannula 110 is engaged by the puncture tool 130 and is driven into the fistula. During this process, the measurement cannula assembly's guide rails 544 encounter the tip 537 of the flexible tubing 536 within the U-shaped channel 538, pushing the refraction mechanism into place in the manner indicated in FIG. 5C.

FIG. 5D is a side view corresponding to portions of a main body 520 of a handheld mechatronic tracheoesophageal puncture and voice prosthesis insertion tool 500 following an insertion stroke, just after voice prosthesis insertion in accordance with an embodiment of the present disclosure. As illustrated in FIG. 5D, a terminal end of the insertion tool external to the handheld tool's main body extends beyond a terminal end of the measurement cannula as a result of the forward motion of the insertion tool and the backward motion of the measurement cannula relative to the main body's length during voice prosthesis insertion and deployment in the patient. During insertion, the force transfer member 540 encounters the retraction mechanism, pushing the flexible tubing 536 along the U-shaped channel 538. The tip(s) 537 of the flexible tubing then push against the measurement cannula assembly's guide rails 544, pulling the measurement cannula 110 out of the fistula.

FIG. 6A is an exploded view of a macro-micro positioning system 600 including an automated TEP end effector 610 configured for carrying a MAID 100 in accordance with an embodiment of the present disclosure. Such a macro-micro positioning system 600 can form portions of a robotic manipulator system, such as an eight degree of freedom (DOF) serial manipulator that includes three passive links and five motorized axles. The manipulator can be mounted on the side rail of a standard surgical bed, or otherwise mounted. The three passive links are responsible for the initial macro positioning of the manipulator, while a five-DOF sub-manipulator system located at a distal end provide the final micro positioning and orientation of the end effector 610. The eight-DOF serial manipulator is given as an example, and other positioning mechanisms can be selected for use in controllable positioning of the TEP end effector, as will be understood by one of ordinary skill in the art.

FIG. 6B is a perspective view of an embodiment of the TEP end effector 610 of FIG. 6A which can be coupled to selected fully or partially automated/computer controllable mechatronic positioning systems. In an embodiment, the effector 610 includes: an end effector interface plate 612; a mini rail linear system 614; a forward mount 616; a latch assembly 618; a MAID 100; a force sensor 620; a force sensor mount 622; and a puncture tool 130. The said end effector 610 has a motor stage 630, allowing for the retraction of the puncture tool and the subsequent insertion of the prosthesis. The force sensor 620 provides puncture detection and braking when TEP is completed. The end effector interface plate 612 can be configured for secure attachment to a controllable positioning system. The motor stage 630 drives the puncture tool and/or MAID along the mini rail linear system 614, which provides improved stability and ensures accuracy of the TEP. The latch assembly 618 provides easy mounting and removal of the MAID before and after the TEP, while the forward mount ensures correct alignment of the MAID relative to the desired path of motion provided by the motor stage. The end effector 610 can include a force sensor 620 secured to a force sensor mount 622, similar to the embodiment described above with respect to FIG. 5A, to provide a built-in safety feature to guard against overextension of the puncture tool 130. The end effector 610 can therefore be a self-contained unit that can be mounted to an existing medical-use robotic arm that a clinic or hospital may already own, making the acquisition of a TEP system according to one embodiment of the present disclosure more affordable to the clinic/hospital and accordingly more affordable to patients.

In an embodiment, a TNE-visualized MAID-based TEP procedure involving an automated or semi-automated TEP creation and voice prosthesis insertion tool includes the following:
(a) Loading the puncture tool, securing with an interface attachment.
(b) using a tip of the puncture tool to palpitate posterior tracheal wall for confirmation of the puncture site through TNE.
(c) Activating a trigger for puncture and waiting for retraction. During the forward stroke of the puncture process, the puncture tool and measurement cannula is propelled through the plastic front cannula, penetrating the posterior tracheal wall and anterior esophageal wall, creating the tracheoesophageal fistula. During the retraction stroke, the measurement cannula is left within the fistula while the puncture tool is removed for replacement with the insertion tool.
(d) Disconnecting the puncture tool and removing puncture tool while maintaining the measurement cannula within the fistula.
(e) Discerning the length of fistula. Voice prosthesis length is selected based on the width of the tracheoesophageal dividing wall. As the motor encoder provides position data, measurement can be performed by subtracting the insertion depth from the marking seen with TNE within the esophagus. Alternatively, in event of slippage, the external as well as intra-luminal markings of the measurement cannula can also be visualized to obtain the appropriate length manually.
(f) Loading the selected voice prosthesis on the insertion tool and securing the insertion tool to the actuator. A single forward stroke pushes the voice prosthesis into place. Simultaneously, the lower half of the insertion tool pushes against the retraction mechanism. The force is transferred along the flexible tubing within the U-channel, pushing against the measurement cannula, in the opposite direction of the insertion tool.
(g) Activating the trigger for prosthesis insertion. The insertion stroke results in the removal of the measurement cannula from the fistula while pushing the prosthesis into place, allowing the prosthesis flanges to expand and engage the anterior esophageal and posterior tracheal wall.
(h) Removing the MAID.

FIG. 6C is a schematic illustration of a prototype automated macro-micro positioning system 600 carrying an automated tracheoesophageal puncture and voice prosthesis insertion end effector 610, and a display screen capture 700 of a computer simulation corresponding thereto in accordance with an embodiment of the present disclosure. Coupled with the force sensing and control capabilities described above, a computer can be programmed to simulate, plan, enable, and/or control computer-aided TEP and voice prosthesis insertion by a robotic arm carrying an end-effector such as one shown in FIG. 6B. The computer can include a processing unit and a memory in which program instructions (e.g., software) reside, which can be executed by the processing unit to provide or display a virtual representation of a tracheoesophageal puncture and voice prosthesis insertion end effector that is mounted to a macro-micro positioning system, the operation of which can be preprogrammed by a user. This advantageously provides the user with limitless opportunities to plan, simulate or try out, and analyze/evaluate different strategies for carrying out the TEP procedure. This enables even a less experienced user to determine optimal needle placement and insertion conditions. According to one embodiment, finite element method codes are integrated in the framework of multi-body dynamics codes to incorporate realistic physical parameters and factors for the representation of the end-effector and robotic arm, as well as a deformable and divisible organ such as the posterior tracheal wall. An object oriented approach is can accommodate a structural description of relatively autonomous objects, each capable of different states and behavior. The heavy computational needs of a medical robotic simulation system can be met by drawing on parallel computing methods.

Aspects of particular embodiments of the present disclosure address at least one aspect, problem, limitation, and/or disadvantage associated with existing systems and techniques for TNE-visualized TEP and voice prosthesis insertion. While features, aspects, and/or advantages associated with certain embodiments have been described in the disclosure, other embodiments may also exhibit such features, aspects, and/or advantages, and not all embodiments need necessarily exhibit such features, aspects, and/or advantages to fall within the scope of the disclosure. It will be appreciated by a person of ordinary skill in the art that several of the above-disclosed systems, components, processes, or alternatives thereof, may be desirably combined into other or different systems, components, processes, and/or applications. For instance, embodiments in accordance with the present disclosure can be configured or utilized for essentially any surgical process or procedure that involves puncture access (e.g., reliably/repeatably controlled puncture access) to a body of an organism, insertion or deployment of a device such as a prosthesis in a body of an organism, and/or bodily insertion or extraction of a material, substance, or composition. As representative non-limiting examples, embodiments in accordance with the present disclosure can be configured for laparoscopic trocar insertion, catheter placement, or biopsy device insertion. In addition, various modifications, alterations, and/or improvements may be made to various embodiments that are disclosed by a person of ordinary skill in the art within the scope and spirit of the present disclosure. Such modifications, alterations, and/or improvements to embodiments in accordance with the present disclosure are encompassed by the following representative claims.

1. A measurement cannula comprising:
a body having a channel therethrough, the body carrying a set of markings including:
a first set of markings detectable by a visual inspection device that is disposable within a body of an organism; and
a second set of markings that is simultaneously visually detectable external to the body of the organism when the measurement cannula is disposed within a fistula formed in the body of the organism.

2. The measurement cannula of claim 1, wherein the first set of markings is detectable by a transnasal esophagoscopy (TNE) device.

3. The measurement cannula of claim 1, wherein the fistula is a tracheoesophageal fistula.

4. The measurement cannula of claim 1, wherein the channel through the body is configured for passage of a voice prosthesis therethrough.

5. A measurement cannula assembly comprising:
an inner cannula; and
an outer cannula configured for mating engagement with the inner cannula, the outer cannula carrying a set of markings detectable by a visual inspection device that is disposable within a body of an organism.

6. The measurement cannula assembly of claim 5, wherein the outer cannula carries a set of markings detectable by a TNE device.

7. The measurement cannula assembly of claim 5, wherein the outer cannula carries a set of markings including a first set of markings detectable by a visual inspection device that is disposable within the body of the organism and a second set of markings that is simultaneously visually detectable external to the body of the organism when the outer cannula is disposed within a fistula formed in the body of the organism.

8. The measurement cannula assembly of claim 5, wherein the outer cannula carries a set of markings including a first set of markings detectable by a visual inspection device that is disposable within the body of the organism and a second set of markings that is simultaneously visually detectable external to the body of the organism when the outer cannula is disposed within a tracheoesophageal fistula.

9. The measurement cannula assembly of claim 5, wherein the outer cannula has an inner diameter configured for passage of a voice prosthesis through the outer cannula.

10. The measurement cannula assembly of claim 5, wherein at least one of the inner cannula and the outer cannula is configured for mating engagement with a small dilator of a Portex mini-tracheostomy kit.

11. A device comprising:
a main body couplable to each of a tissue puncture tool and a measurement cannula, the measurement cannula including a first set of markings detectable by a visual inspection device that is disposable within a body of an organism; and
an actuator configured to impart a displacement force to at least one of the tissue puncture tool and the measurement cannula to thereby drive each of the tissue puncture tool and the measurement cannula in a common direction along a single axis during a tissue puncture stroke during which each of the puncture tool and the cannula can be driven into the body of the organism.

12. The device of claim 11, wherein the measurement cannula is configured for mating engagement with the tissue puncture tool.

13. The device of claim 11, wherein the measurement cannula includes a second set of markings that is visually detectable external to the body of the organism when the first set of markings is disposed internal to the body of the organism.

14. The device of claim 11, wherein the first set of markings is detectable by a transnasal esophagoscopy (TNE) device.

15. The device of claim 11, wherein the actuator is configured to impart a displacement force that causes tissue puncture tool displacement at a rate of at least approximately 2 mm/s.

16. The device of claim 11, wherein the actuator is configured to impart a displacement force that causes tissue puncture tool displacement at a rate of between approximately 7.5 mm/s and approximately 12.5 mm/s.

17. The device of claim 11, further comprising a set of sensors configured to detect when the tissue puncture tool has exited bodily tissue and entered a target bodily lumen.

18. The device of claim 17, further comprising a feedback mechanism coupled to the set of sensors and the actuator for terminating the puncture stroke in response to the tissue puncture tool exiting bodily tissue and entering the target bodily lumen.

19. The device of claim 11, wherein the main body is further couplable to a prosthesis insertion tool, and wherein the main body includes a retraction mechanism configured for displacing the measurement cannula in a direction opposite to a direction of travel of the prosthesis insertion tool during a prosthesis insertion stroke during which the actuator imparts a displacement force that causes the prosthesis insertion tool to be driven into the body of the organism.

20. The device of claim 19, wherein the prosthesis insertion tool is configured to travel within a channel formed in the measurement cannula during the prosthesis insertion stroke.

21. A device comprising:
a body comprising;
a channel configured for carrying each of a tissue puncture tool, a measurement cannula, and a prosthesis insertion tool along a single common axis;
an actuator configured to impart a first displacement force to the tissue puncture tool causing the tissue puncture tool and the measurement cannula to travel in a first direction along the single common axis and a second displacement force to the prosthesis insertion tool causing the prosthesis insertion tool to travel in the first direction; and
a retraction mechanism configured for selectively engaging the measurement cannula causing the measurement cannula to travel in a second direction opposite to the first direction during prosthesis insertion tool travel in the first direction.

22. The device of claim 21, wherein the measurement cannula includes a channel formed therein configured to matingly receive the tissue puncture tool.

23. The device of claim 21, wherein the measurement cannula includes a channel formed therein configured for passage of a portion of the prosthesis insertion tool through the measurement cannula.
